(19) United States
(12) Patent Application Publication
(10) Pub. No.: US 2025/0032791 A1
(43) Pub. Date: Jan. 30, 2025

(54) APPARATUS AND METHOD OF REDUCING SEVERITY OF SYMPTOMS OF DEGENERATIVE BRAIN DISEASE

(71) Applicant: HEBY, LLC, Hopewell, NJ (US)

(72) Inventors: Yakov KATSNELSON, Sudbury, MA (US); Hank BECKHOFF, Perkasie, PA (US); Vladimir CHUEV, St. Petersberg (RU)

(73) Assignee: HEBY, LLC, Hopewell, NJ (US)

(21) Appl. No.: 18/782,185

(22) Filed: Jul. 24, 2024

Related U.S. Application Data
(60) Provisional application No. 63/528,653, filed on Jul. 25, 2023.

(51) Int. Cl. A61N 1/36; A61N 1/02; A61N 1/04

(52) U.S. Cl. CPC A61N 1/36034; A61N 1/025; A61N 1/0408; A61N 1/0476; A61N 1/36025

(57) ABSTRACT

The present disclosure provides for a multichannel device configured to provide transcranial electrostimulation with direct and interference currents using a migrating anode. Such transcranial electrostimulation helps to prevent acquired tolerance and/or tachyphylaxis in a user.

### CROSS-REFERENCE TO RELATED APPLICATIONS

This application claims priority to U.S. Application Ser. No. 63/528,653, filed on Jul. 25, 2023, the contents of which are herein fully incorporated by reference in its entirety.

### BACKGROUND OF THE EMBODIMENTS

Transcranial direct current stimulation (TDCS) is a noninvasive brain stimulation technique that is thought to improve cognitive impairment in patients with mild cognitive impairment (MCI) and Alzheimer's disease (AD). As a result, TDCS has received increasing attention as of late.

Generally, TDCS can be divided into anodal (anodic) and cathodal (cathodic) methods of stimulation. Anodic TDCS serves to depolarize neuronal resting potential and increase cortical excitability by increasing the frequency of spontaneous neuronal firing, while cathodic TDCS serves to hyperpolarize neuronal resting potential and suppress cortical excitability by reducing neuronal firing frequency.

However, it has also been observed that compared to cathodic stimulation with a current of 1 mA, cathodic stimulation with a current of 2 mA can increase cortical excitability. Changes in cortical excitability induced by TDCS may lead to corresponding changes in cortical function and activation. Increased cortical excitability and, in turn, neuroplasticity are important mechanisms for improving clinical and cognitive abilities in neurodegenerative diseases such as MCI and AD. In addition, studies have suggested that the improvement in cognitive function in TDCS may be related to the neural noise produced by TDCS. Further studies have shown that TDCS can have varying degrees of therapeutic effect on various neurodegenerative diseases, including Parkinson's disease, AD, and primary progressive aphasia and gate control as well as potentially improving motor function in patients with Parkinson's disease.

While the benefits of TDCS are apparent, repeated administration of TDCS can lead to the development of the effect of an acquired tolerance. As understood in this context, “tolerance” is the requirement of higher doses to produce a given response or the reduction in response after repeated administrations. Tolerance can lead to a decrease in the effectiveness TDCS. To combat tolerance, the modus operandi is to increase the dosage of medications or to increase the applied current amplitude to the user.

To prevent the development of tolerance, and without the need to increase the amplitude of the current, the present invention and its embodiments propose the use of a migrated anode, which would reduce the likelihood of developing tolerance.

### SUMMARY OF THE EMBODIMENTS

In general, the present invention and its embodiments provide for a multichannel device configured to provide transcranial electrostimulation with direct and interference currents using a migrating anode.

In one aspect of the present invention, there is a method of transcranial stimulation, the method includes the steps of providing at least three pairs of electrodes, using a controller coupled to the at least three pairs of electrodes to activate or deactivate the at least three pairs of electrodes thereby causing at least one of each of the at least three pairs of electrodes to emit one or more electronic pulses, where the one or more electronic pulses includes a carrier wave having a first frequency, and where the carrier wave is modulated by a modulating frequency to create a modulated wave. The method also includes a multiplexer communicatively coupled to the controller configured to provide anode migration.

The method may also include one or more current sources coupled to the multiplexer.

The method may also include where a first pair of the at least three pairs of electrodes are coupled to a forehead of a user.

The method may also include where a second pair of the at least three pairs of electrodes are coupled to a temporal region of a user.

The method may also include where a third pair of the at least three pairs of electrodes are coupled to a neck of a user.

The method may also include where a fourth pair of the at least three pairs of electrodes are coupled to a mastoideus region of a user.

The method may also include where the one or more electronic pulses comprise at least one of a direct current and an interference current. Other technical features may be readily apparent to one skilled in the art from the following figures, descriptions, and claims.

The method may also include where the anode migration occurs at least one every five minutes.

The method may also include where the one or more electronic pulses change from the direct current and the interference current at least once every thirty minutes.

In another aspect of the present invention, there is a method of transcranial stimulation, the method includes the steps of providing at least three pairs of electrodes coupled to a user, using a controller coupled to the at least three pairs of electrodes to activate or deactivate the at least three pairs of electrodes thereby causing at least one of each of the at least three pairs of electrodes to emit one or more electronic pulses, where the one or more electronic pulses includes a carrier wave having a first frequency, where the carrier wave is modulated by a modulating frequency to create a modulated wave, and where the one or more electronic pulses provide a direct current for a first time period and an interference current for a second time period. The method also includes a multiplexer communicatively coupled to the controller configured to provide anode migration.

The method may also include where a polarity of the one or more electronic pulses is periodically inverted.

The method may also include where when the one or more electronic pulses are configured to provide the direct current in a range of about 2.0 to about 4.5 mA.

The method may also include where when the one or more electronic pulses are configured to provide the interference current in a range of about 500 kHz to about 2 mHz. Other technical features may be readily apparent to one skilled in the art from the following figures, descriptions, and claims.

In yet another aspect of the present invention there is a method of transcranial stimulation, the method includes the steps of providing at least three pairs of electrodes coupled to a user, where the at least three pairs of electrodes are coupled to a forehead, a neck, a temporal region, and a mastoideus region of the user, using a controller coupled to the at least three pairs of electrodes to activate or deactivate the at least three pairs of electrodes thereby causing at least one of each of the at least three pairs of electrodes to emit one or more electronic pulses, where the one or more electronic pulses includes a carrier wave having a first frequency, where the carrier wave is modulated by a modulating frequency to create a modulated wave, and where the one or more electronic pulses provide a direct current for a first time period and an interference current for a second time period. The method also includes a multiplexer communicatively coupled to the controller configured to provide anode migration, where the anode migration occurs once about every five minutes of the first time period and the second time period.

The method may also include where there are three current sources coupled to the multiplexer.

The method may also include where a position of the at least three pairs of electrodes is the same for an application of the direct current or the interference current to the user.

The method may also include where the interference current is modulated in a range of about 28 Hz to 33 Hz and 48 to 53 Hz.

In general, the present invention succeeds in conferring the following benefits and objectives.

It is an object of the present invention to provide a TDCS device that is lightweight and easy to use.

It is an object of the present invention to provide a TDCS device that reduces or alleviates the severity of symptoms of degenerative brain diseases.

It is an object of the present invention to provide a TDCS device that is non-invasive.

It is an object of the present invention to provide a TDCS device that improves or alleviates symptoms associated with mild cognitive impairment and Alzheimer's disease.

It is an object of the present invention to provide a TDCS device that reduces or alleviates the effects of acquired tolerance and/or tachyphylaxis.

It is an object of the present invention to provide a TDCS device that does not require one to increase the amplitude of the current applied to the user.

Other technical features may be readily apparent to one skilled in the art from the following figures, descriptions, and claims.

### BRIEF DESCRIPTION OF THE DRAWINGS

To easily identify the discussion of any particular element or act, the most significant digit or digits in a reference number refer to the figure number in which that element is first introduced.

FIG. 1 illustrates an embodiment of a multichannel device using a migrating anode.

FIG. 2 illustrates a multiplexer in accordance with an embodiment of the multichannel device.

FIG. 3A illustrates the placement of the electrodes of the multichannel device on the left side of a head of a user.

FIG. 3B illustrates the placement of the electrodes of the multichannel device on the right side of a head of a user.

FIG. 4A is a diagram illustrating a bipolar carrier frequency modulated by a modulating frequency in accordance with an embodiment of the present invention.

FIG. 4B is another diagram illustrating a bipolar carrier frequency modulated by a modulating frequency in accordance with an embodiment of the present invention.

FIG. 5 is a diagram illustrating oscillations from the modulated carrier frequency in accordance with an embodiment of the present invention.

### DETAILED DESCRIPTION OF THE EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

For the purposes of this application, “about” is intended to mean the value or values provided as well as a variance of up to −10% to +10% for any given value. For example, about 100 means any number from 90 to 110 and includes 100. Further, whole numbers and fractional numbers (90.2, 90.3, etc.) are included within this definition.

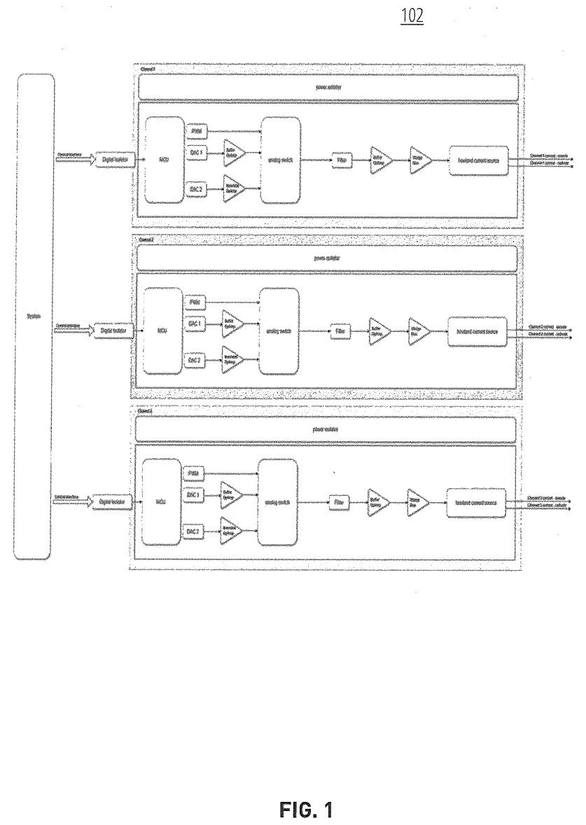

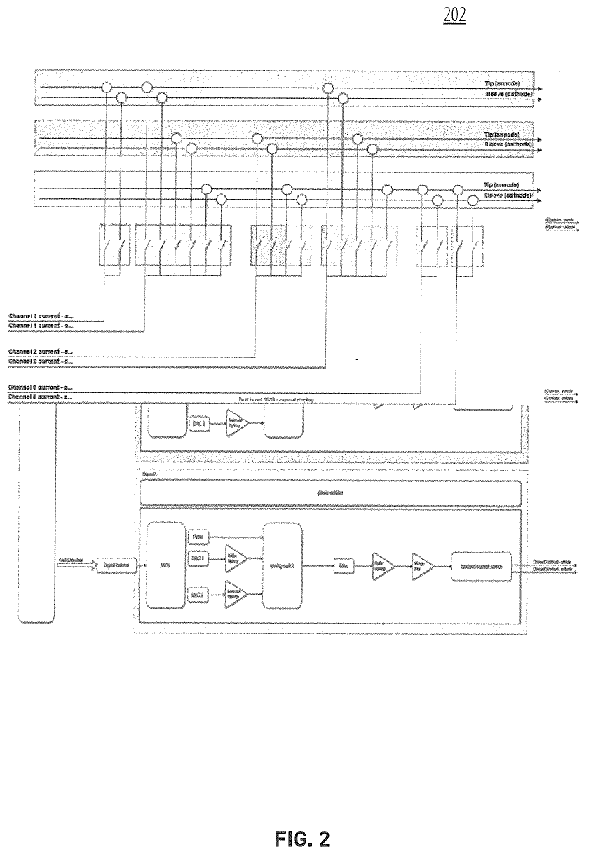

Referring now to FIG. 1, there is a schematic showing the general components of a transcranial stimulation device 102 consistent with an embodiment of the present invention. Included are at least a control interface, electrodes, digital isolator, microcontroller, pulse-width modulator, inversion operational amplifier, analog switch, voltage-controlled current source, an anode/cathode migration module (see FIG. 2), and a power isolator.

Referring now to FIG. 2, there is an anode/cathode migration module comprising a multiplexer 202. The cathode/anode migration module may use up to at least three (3) current sources which are connected via a controlled multiplexer 202 to the electrode sockets. In some embodiments, the anode and cathode can be swapped, or one anode/cathode can be connected to more than one electrode. As a complete signal rearrangement is not required to maintain the anode/cathode migration, the multiplexer 202 has been optimized to get rid of unused connection options.

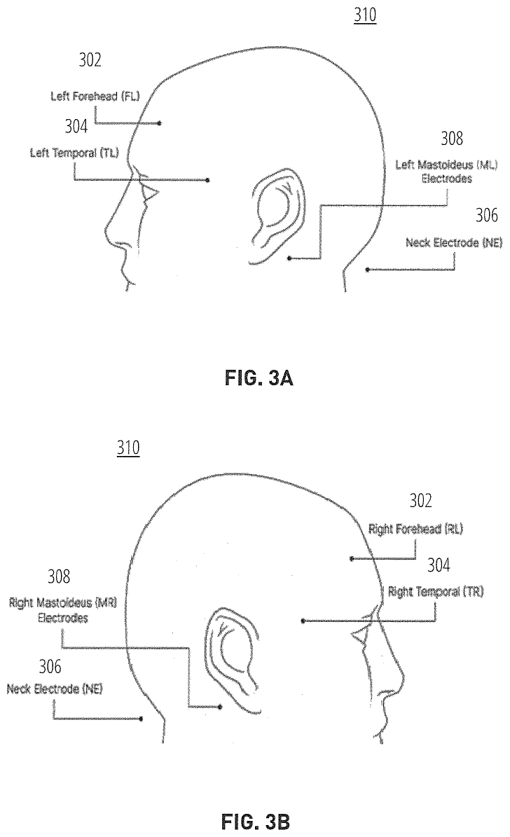

FIGS. 3A and 3B demonstrate the arrangement of the electrodes in at least one embodiment of the present invention. Shown is a user 310 from a first or left profile view (FIG. 3A) and from a second or right profile view (FIG. 3B). The terms “patient,” “subject,” “user,” or “host” as used herein may mean either a human or non-human animal, such as primates, mammals, and vertebrates. Preferably, a user 310 is a human.

The electrodes used to deliver the one or more electronic pulses consistent with the embodiments of the present invention are electrodes generally known in the art. Such electrodes may be selectively adhered to a skin surface of the user 310. As shown, the first pair of the at least three pairs of electrodes 302 are coupled to a left forehead (FL) area and a right forehead (FR) area of the user 310. As shown, the second pair of the at least three pairs of electrodes 304 are coupled to a left temporal region (TL) and a right temporal region (TR) of the user 310. As shown, the third pair of the at least three pairs of electrodes 306 are coupled to a rear neck area (NE) of the user 310. In some embodiments, however, the third pair of the at least three pairs of electrodes 306 only comprises one electrode coupled to a rear neck area of the user 310. As shown, the fourth pair of the at least three pairs of electrodes 308 are coupled to a left mastoideus (ML) region and a right mastoideus region (MR) of the user 310. The general placement of the electrodes on the skin surface of the user 310 are critical to the embodiments of the present invention.

The proposed electrode arrangement, as described with respect to FIGS. 3A and 3B, and algorithm of their combinations during stimulation provide both anodal transcranial direct current and cathodal transcranial direct current stimulation. This, without being bound by theory, is intended to provide the development of important mechanisms that improve clinical and cognitive abilities in neurodegenerative diseases. In a preferred embodiment, the transcranial direct current stimulation will initially be applied to a user. Preferably, the transcranial direct current stimulation is applied in a range of about 0.1 mA to about 10 mA and more preferably in a range of about 1 mA to about 7 mA and most preferably in a range of about 2 mA to about 4.5 mA. It is envisioned that the anode migration (as described in FIG. 2) will occur at least one about every five (5) minutes. In at least one embodiment, the algorithm of anode migration follows at least one of the below patterns.
A. The anode—right forehead (FR), left forehead (FL); the cathode—neck (NE), right temporal region (TR), left temporal region (TL) and right mastoideus region (MR), left mastoideus region (ML)
B. 1—The anode—TR; the cathode—TL
   2—The anode—MR; the cathode—ML
   3—The anode—NE; the cathode—FR, FL
C. 1—The anode—TL; the cathode—TR
   2—The anode—ML; the cathode—MR
   3—The anode—FR, FL; the cathode—NE
D. The anode—TR and MR; the cathode—FR, FL, NE, ML, and TL
E. The anode—NE; the cathode—FR, FL, TR, TL, MR, and ML
F. The anode—TL and ML; the cathode—FR, FL, NE, TR, and MR

After about thirty (30) minutes of the direct current transcranial stimulation, the direct current will change to a transcranial interference current stimulation as further described herein.

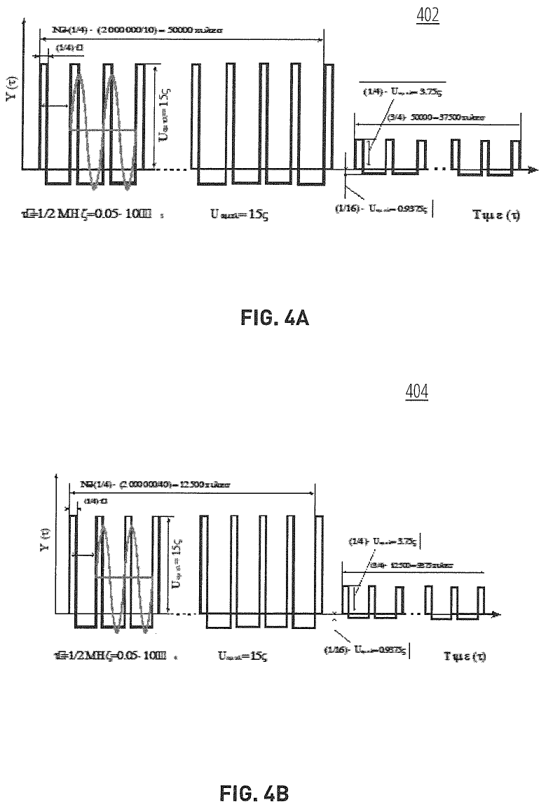

Referring now to FIGS. 4A and 4B of the present application, there is a first waveform 402 and a second waveform 404 respectively. In each are diagrams illustrating a bipolar carrier frequency being modulated by a modulating frequency of about 10 Hz (FIG. 4A) and about 40 Hz (FIG. 4B). The use of the interference effect occurring in the deep structures of the brain makes it possible to achieve stimulation of these structures noninvasively, without the need to implant electrodes and thus avoid the development of side-effects associated with surgical intervention. Further, the proposed algorithm of transcranial stimulation allows for the formation of two or more mutually perpendicular alternating currents in the brain, each interfering with the other to achieve the desired effects.

The low-frequency dielectric constant of the human brain is e=2860. The speed of an electromagnetic wave in the substance of the brain is calculated according to the equation

$$V = C / n = C / {( e )}^{1 / 2} = ( 3.1 {0}^{8} ) / ( 54 ) = 6 {\text{.10}}^{6} M / C = 6000 km / s$$

Thus, the electromagnetic wavelength in the proposed diapason in the brain is calculated according to the equations below:

$$1 = V / f = ( 6 \; 000 \; 000 ) / ( 20 \; 00 \; 000 ) = 3 m$$

$$1 = V / f = ( 6 \; 000 \; 000 ) / ( 300 \; 000 ) = 20 m$$

Thus, the length of the electromagnetic wave in the brain is in diapason from about 3 m to 20 m with a frequency of electromagnetic oscillations at about 300 kHz to about 2 mHz. The arrangement of pairs of electrodes, in accordance with the present invention, via anode migration is such that the distance between sources of electromagnetic waves emitted by the electrodes is small; much less than the length of the electromagnetic wave in the substance of the brain. Therefore, the electromagnetic oscillations of both sources occur in the same phase. That is, the phase difference of oscillations between them is zero. This means that both oscillations simultaneously reach a maximum, simultaneously pass through zero and at the same time reach a minimum. Therefore, with the interference of waves propagated in the brain substance by emitters at 300 kHz-2 mHz with zero phase difference, created the simple mutual increase in amplitude of pulses.

Without the distortion of the pulse shape and without changing the frequency, such pulsed oscillations with a frequency of about 300 kHz to about 2 mHz can, of course, be decomposed into the Fourier spectrum with frequencies that are multiples of about 300 kHz to about 2 mHz. However, all Fourier components of different frequencies do not undergo distortion for the reason(s) described herein. Therefore, the frequencies are added to each other in the interference zone without phase shift. After the addition, in the interference zone, the frequencies create undistorted pulsed oscillations, doubling the amplitude of each Fourier component.

A fundamentally different situation exists for oscillations modulating the carrier frequency. Both radiators of modulating oscillations oscillate with different frequencies and propagate in the substance of the brain at different speeds. Therefore, between the modulating oscillations, there is initially a phase difference, the magnitude of which varies periodically in the interference zone. For this reason, in the interference zone occur carrier frequency oscillations (about 500 kHz to about 2 mHz), which are modulated in a complex manner. In this complex oscillation, there are two frequencies equal to the half-sum and half-difference of the modulating frequencies. Such holds true for the main sine-wave Fourier harmonic of each modulating pulse oscillation. The view of the main harmonic is presented in both FIGS. 4A and 4B. Higher Fourier harmonics do not conventionally play a large role in the described process, since their amplitudes are insignificant given the size.

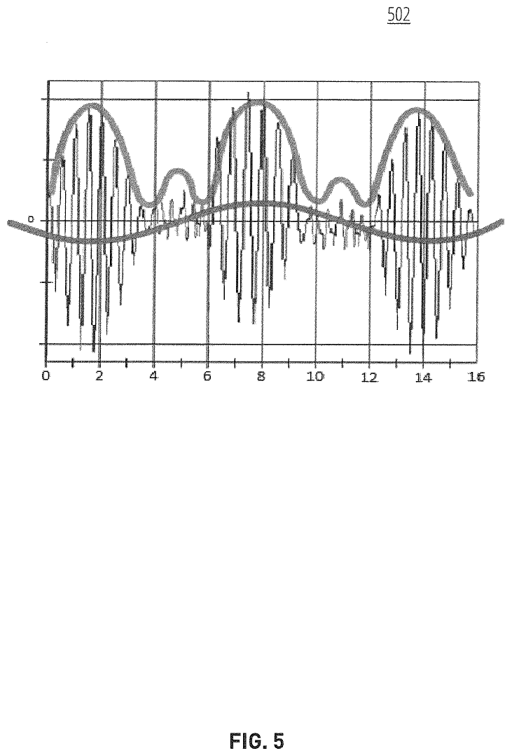

FIG. 5 illustrates an oscillation modulating carrier frequency 502 featuring the high-frequency oscillation of the carrier frequency as well as two other oscillations with differing frequencies. One of the frequencies is approximately equal to the half-sum frequency of the main Fourier harmonics of the modulating oscillations. For 25 Hz and 15 Hz this gives a value of about 20 Hz. The other frequency is equal to the half-difference of these frequencies, and for 25 Hz and 15 Hz it is about 5 Hz. It follows that to obtain 40 Hz and 10 Hz, a doubling of frequencies is required, thereby modulating the carrier from about 500 kHz-to about 2 mHz. This necessitates the use of the modulating frequency of 50 Hz and 30 Hz. Based on the drawings and information presented herein, and without being bound by theory, a comparison was made of 25 and 15 Hz and 50 and 30 Hz, used to obtain modulating frequencies of 40 and 10 Hz.

It is preferred that the interference pulses will be applied to a user consistent with the following parameters. A carrier frequency having bipolar impulses with a frequency of about 300 kHz to about 2 mHz. Further, the implementation has frequency changes randomly every one hundred and twenty (120) seconds, in steps of about 100 kHz with a permissible deviation of no more than 10%. In a preferred embodiment, a duty cycle for high amplitude is about 20% of the pulse and a duty cycle for low amplitude is about 80% of the pulse. Further, the interference pulses experience polarity changes every about every six hundred (600) seconds which occurs over a thirty (30) second polarity reversal time (e.g., 15 seconds of current decreases to zero, and 15 seconds of current is set to the value preceding the change in polarity).

For the modulation frequencies for the interference pulses, the following parameters are preferred: the modulation depth is 75%; a duty cycle for high amplitude is about 20% of the pulse; a duty cycle for low amplitude is about 80% of the pulse; a correlation between high amplitude and low amplitude pulse is 4; and the anode for modulating frequencies migrates according to a preferred embodiment about once every five (5) minutes.

### EXAMPLE

Channels of stimulation
Channel 1—48-53 Hz: the anode—FR, FL; and the cathode—NE
Channel 2—26-35 Hz: the anode—TL; and the cathode—TR
Channel 3—26-35 Hz: the anode—ML; and the cathode—MR
Channel 4—48-53 Hz: the anode—NE; and the cathode—FR, FL
Channel 5—26-35 Hz: the anode—TR; and the cathode—TL
Channel 6—26-35 Hz: the anode—MR; and the cathode—ML

Potential combination of the channels described above:
Channel 1, Channel 2, Channel 3
Channel 4, Channel 5, Channel 6
Channel 1, Channel 5, Channel 6
Channel 4, Channel 2, Channel 3

In a preferred embodiment, the increase in current and its decrease during the beginning and the end of stimulation, and during anode migration occurs within fifteen (15) seconds, the duration of the increase in current and its decrease during the beginning and the end of stimulation, and during of anode migration is not considered in the time of treatment. In a preferred embodiment, a user may repeat the entire cycle (TDCS and interference current) about two (2) to three (3) times and on average duration of stimulation is thirty (30) minutes of direct current stimulation and thirty (30) minutes of interference stimulation.

Although this invention has been described with a certain degree of particularity, it is to be understood that the present disclosure has been made only by way of illustration and that numerous changes in the details of construction and arrangement of parts may be resorted to without departing from the spirit and the scope of the invention.

What is claimed is:

1. A method of transcranial stimulation, the method comprising the steps of:
providing at least three pairs of electrodes;
using a controller coupled to the at least three pairs of electrodes to activate or deactivate the at least three pairs of electrodes thereby causing at least one of each of the at least three pairs of electrodes to emit one or more electronic pulses,
wherein the one or more electronic pulses comprises a carrier wave having a first frequency, and
wherein the carrier wave is modulated by a modulating frequency to create a modulated wave; and
a multiplexer communicatively coupled to the controller configured to provide anode migration.

2. The method of claim 1 further comprising one or more current sources coupled to the multiplexer.

3. The method of claim 2 wherein there are three current sources coupled to the multiplexer.

4. The method of claim 1 wherein a first pair of the at least three pairs of electrodes are coupled to a forehead of a user.

5. The method of claim 1 wherein a second pair of the at least three pairs of electrodes are coupled to a temporal region of a user.

6. The method of claim 1 wherein a third pair of the at least three pairs of electrodes are coupled to a neck of a user.

7. The method of claim 1 wherein a fourth pair of the at least three pairs of electrodes are coupled to a mastoideus region of a user.

8. The method of claim 1 wherein the one or more electronic pulses comprise at least one of a direct current and an interference current.

9. The method of claim 8 wherein a position of the at least three pairs of electrodes is the same for an application of the direct current or the interference current to the user.

10. The method of claim 1 wherein the anode migration occurs at least one every five minutes.

11. The method of claim 8 wherein the one or more electronic pulses change from the direct current and the interference current at least one every thirty minutes.

12. A method of transcranial stimulation, the method comprising the steps of:
providing at least three pairs of electrodes coupled to a user;
using a controller coupled to the at least three pairs of electrodes to activate or deactivate the at least three pairs of electrodes thereby causing at least one of each of the at least three pairs of electrodes to emit one or more electronic pulses,
wherein the one or more electronic pulses comprises a carrier wave having a first frequency,
wherein the carrier wave is modulated by a modulating frequency to create a modulated wave, and
wherein the one or more electronic pulses provide a direct current for a first time period and an interference current for a second time period; and
a multiplexer communicatively coupled to the controller configured to provide anode migration.

13. The method of claim 12 wherein a polarity of the one or more electronic pulses is periodically inverted.

14. The method of claim 12 wherein when the one or more electronic pulses are configured to provide the direct current in a range of about 2.0 to about 4.5 mA.

15. The method of claim 12 wherein when the one or more electronic pulses are configured to provide the interference current in a range of about 500 kHz to about 2 mHz.

16. The method of claim 15 wherein the interference current is modulated in a range of about 28 to 33 Hz and 48 to 53 Hz.

17. A method of transcranial stimulation, the method comprising the steps of:
providing at least three pairs of electrodes coupled to a user,
wherein the at least three pairs of electrodes are coupled to a forehead, a neck, a temporal region, and a mastoideus region of the user;
using a controller coupled to the at least three pairs of electrodes to activate or deactivate the at least three pairs of electrodes thereby causing at least one of each of the at least three pairs of electrodes to emit one or more electronic pulses,
wherein the one or more electronic pulses comprises a carrier wave having a first frequency,
wherein the carrier wave is modulated by a modulating frequency to create a modulated wave, and
wherein the one or more electronic pulses provide a direct current for a first time period and an interference current for a second time period; and
a multiplexer communicatively coupled to the controller configured to provide anode migration,
wherein the anode migration occurs once about every five minutes of the first time period and the second time period.
